(19) United States
(12) Patent Application Publication
(10) Pub. No.: US 2013/0131710 A1
(43) Pub. Date: May 23, 2013

(54) DEVICE SYSTEM AND METHOD FOR RESHAPING TISSUE OPENINGS

(71) Applicants: Ran Carmeli, Moshav Rinatya (IL); Eyal Teichman, Hod-HaSharon (IL); Guy Kotlizky, Kfar-Shemaryahu (IL)

(72) Inventors: Ran Carmeli, Moshav Rinatya (IL); Eyal Teichman, Hod-HaSharon (IL); Guy Kotlizky, Kfar-Shemaryahu (IL)

(73) Assignee: Assis Medical Ltd., Herzlia Pituach (IL)

(21) Appl. No.: 13/521,226

(22) PCT Filed: Jan. 3, 2011
(86) PCT No.: PCT/IL11/00002

§ 371 Date: Jul. 10, 2012

Related U.S. Application Data
(60) Provisional application No. 61/293,748, filed on Jan. 11, 2010.

(51) Int. Cl. A61M 29/02

(52) U.S. Cl. CPC A61M 29/02
USPC 606/194

(57) ABSTRACT

A device for resizing/reshaping a tissue opening is provided. The device includes a cage structure which is positionable within the tissue opening and is radially expandable therein while enabling flow therethrough while it is fully expanded and biased against a tissue defining the tissue opening.

### FIELD AND BACKGROUND OF THE INVENTION

The present invention relates to device for reshaping tissue opening such as a valve orifice and a system and method utilizing same. In one embodiment, the present device is designed for performing valvuloplasty procedures.

Heart valve abnormalities such as valvular insufficiency and valvular stenosis can result in insufficient opening or closure of a heart valve. Valvular insufficiency is a common cardiac abnormality where the valve leaflets do not completely close. This allows regurgitation (i.e., backward leakage of blood at a heart valve). Such regurgitation requires the heart to pump both the regular volume of blood and the blood that has regurgitated. Such added workload can eventually result in heart failure.

Valvular stenosis or calcification is a calcium buildup in the valve which impedes proper valve leaflet movement and can severely limit opening of the valve.

Traditionally, heart valve abnormalities are treated via open heart surgery, however, in individuals whose heart function is too severely compromised to withstand surgery; percutaneous approaches for treating heart valve disease have been developed.

Percutaneous valvotomy (also called valvuloplasty) is typically performed to treat mitral valve and pulmonic valve stenosis; in some patients it may also be performed to treat stenosis of the aortic valve.

Percutaneous valve replacement (PVR) is a procedure for pecutaneously replacing a heart valve. It is performed by placing a catheter through the femoral artery (in the groin) or through a radial artery and guiding it into the chambers of the heart. A compressed tissue heart valve is placed on the balloon-mounted catheter and is positioned directly over the diseased aortic valve. Once in position, the balloon is inflated to secure the valve in place.

Valvuloplasty is a procedure originally developed for treating stenotic heart valves. A catheter is advanced from a blood vessel in the groin through the aorta into the heart. Once the catheter is placed in the stenotic valve, a balloon mounted at the tip of the catheter is inflated until the leaflets (flaps) of the valve are forced open. The balloon is then deflated and the catheter is removed. Although this procedure can be effective in treatment of mitral and pulmonic valve stenosis, it is not considered effective in treatment of severe symptomatic aortic stenosis.

In recent years, valvuloplasty has been adapted as a first step in percutaneous valve replacement procedures since it has been postulated that reshaping of stenotic heart valves prior to placement of the prosthetic valve can improve the outcome of PVR.

Although currently used valvuloplasty balloons are somewhat effective in reshaping valve tissue, they suffer from several inherent limitations.

Since inflated balloon are elastic in nature, they can conform to the shape of the stenosed valve, and as a result, they can be less effective in reshaping severly stenosed valves. Furthermore, balloon valvuloplasty devices completely occlude the valve orifice when in use and as such stop the flow of blood during valve tissue manipulation. This severely limits the time of the procedure and further places an already debilitated patient at risk thereby limiting the patient population on whom the procedure can be performed.

Thus, the present inventors believe that there remains a need for a percutaneous valve preparation system which can be used by a physician to prepare the valve prior to a subsequent valve replacement procedure without the aforementioned limitations.

### SUMMARY OF THE INVENTION

According to one aspect of the present invention there is provided a device for resizing/reshaping a tissue opening comprising a cage structure being positionable within the tissue opening and being radially expandable therein via inflation of at least one balloon positioned within the cage structure, wherein the device enables flow of a biological fluid through the tissue opening when the cage structure is fully expanded and biased against a tissue defining the tissue opening.

According to further features in preferred embodiments of the invention described below, the tissue opening is a heart valve orifice and the biological fluid is blood.

According to still further features in the described preferred embodiments the cage structure is torpedo shaped.

According to still further features in the described preferred embodiments the at least one balloon includes two balloons each positioned within an end portion of the torpedo shaped cage structure.

According to still further features in the described preferred embodiments the cage structure is radially expandable to a diameter of 20-40 mm.

According to still further features in the described preferred embodiments the device further comprises a valve for controlling flow of the biological fluid through the device.

According to still further features in the described preferred embodiments the at least one balloon is configured for reducing drag forces on flow of the biological fluid in one direction, while increasing the drag forces on the flow of the biological fluid in an opposite direction.

According to still further features in the described preferred embodiments the device is designed for compensating for changes in a length of the cage structure during radial expansion thereof.

According to still further features in the described preferred embodiments the cage structure includes elastic struts configured for compensating for changes in the length of the cage structure during radial expansion thereof.

According to still further features in the described preferred embodiments the cage structure is formed of longitudinal struts.

According to another aspect of the present invention there is provided device for resizing/reshaping a tissue opening comprising a cage structure being positionable within the tissue opening and being capable of a first radial expansion via an elastic structural change and a second radial expansion via mechanical expansion.

According to still further features in the described preferred embodiments the elastic structural change is effected by releasing a restraint maintaining the cage structure in a compressed state.

According to still further features in the described preferred embodiments the second radial expansion is effected by a mechanism being capable of radially expanding the cage structure without elastic deformation.

According to still further features in the described preferred embodiments the mechanism includes at least one balloon disposed within the cage structure.

According to still further features in the described preferred embodiments the device enables flow of a biological fluid through the tissue opening when the cage structure is fully expanded and biased against a tissue defining the tissue opening.

According to still further features in the described preferred embodiments the first radial expansion of the cage structure is up to a diameter of 20 mm.

According to still further features in the described preferred embodiments the second radial expansion of the cage structure is up to a diameter of 40 mm.

According to another aspect of the present invention there is provided a device for resizing/reshaping a tissue opening of a body comprising: (a) a device body being positionable within the tissue opening and being radially expandable therein while enabling flow of a biological fluid therethrough when fully expanded and biased against a tissue of the tissue opening; and (b) a valve for controlling flow of the biological fluid through the device.

According to another aspect of the present invention there is provided device for resizing/reshaping a tissue opening of a body comprising: (a) a cage structure being positionable within the tissue opening and being radially expandable therein while enabling flow of a biological fluid therethrough when fully expanded and biased against a tissue of the tissue opening; and (b) a mechanism for compensating for changes in a length of the cage structure during radial expansion thereof.

According to still further features in the described preferred embodiments the mechanism is an elastic deformation of a portion of the cage structure.

According to another aspect of the present invention there is provided a method of resizing/reshaping a tissue opening comprising: (a) positioning a cage structure within the tissue opening; (b) expanding the cage structure within the tissue opening thereby biasing outward tissue defining the tissue opening while enabling biological fluid to flow through the tissue opening, thereby resizing/reshaping the tissue opening; and (c) controlling the flow through the cage structure.

According to another aspect of the present invention there is provided a system for resizing a tissue opening comprising a catheter for delivering the device described herein.

According to still further features in the described preferred embodiments the catheter is configured for percutaneous delivery.

According to still further features in the described preferred embodiments the catheter is configured for trans-apical delivery.

The present invention successfully addresses the shortcomings of the presently known configurations by providing devices systems and methods for resizing/reshaping a tissue opening such as a heart valve while maintaining the tissue opening partially open to flow through of a biological fluid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B illustrate one embodiment of the device of the present invention in side (FIG. 1a) and perspective (FIG. 1b) views.

FIG. 2 illustrates a side view of another embodiment of the device of the present invention.

FIG. 3 illustrates an embodiment of the present device which is designed for compensating for changes in device length during expansion.

FIGS. 4A-D illustrate another embodiment of the device of the present invention, showing the device prior to (FIG. 4a) and following (FIGS. 4b and c) the first expansion phase and following the second expansion stage (FIG. 4d).

### DESCRIPTION OF THE PREFERRED EMBODIMENTS

The present invention is of a device system and method which can be used to resize and/or reshape tissue openings. Specifically, the present invention can be used to perform valvuloplasty procedures on stenotic heart valves and in particular on a stenotic aortic valve.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

A heart valve is composed of an annulus which supports three thin and pliable leaflets in a tricuspid valve, or sometimes two leaflets in a bicuspid valve. In the case of an aortic valve, normal aortic valve leaflets spread apart easily and cause no obstruction to outflow of the blood from the heart. In valve replacement therapy, leaflet functionality is replaced by a prosthetic device which is anchored to annulus tissue and as such, correct sizing of the annulus is a prerequisite step in this procedure.

Devices suitable for manipulating the size and shape of a heart valve are known in the art; such devices are typically referred to as valvuloplasty devices.

Valvuloplasty devices typically employ a catheter carrying a balloon which is positionable within a valve orifice and is inflatable therein to force stenotic valve leaflets open. Some valvuloplasty device employ additional scoring (cutting) elements for cutting stenotic valve leaflets thereby further facilitating reshaping and resizing.

Numerous valvuloplasty devices which employ inflatable balloons for forcing open stenotic heart valve leaflets are known in the art. Although balloons can be used to reshape heart valves, they are less effective in reshaping severely stenosed valves and in addition, they completely occlude the valve orifice when in use and as such stop the flow of blood during valve tissue manipulation.

Several attempts have been made to address these limitations of balloon-type valvuloplasty devices. For example, U.S. 20070118214 describes a braided structure that is expanded using wire-actuated mechanism in order to force open stenotic valves. The advantages of such a device is in that it does not block the flow of blood when in use.

Although U.S. 20070118214 also mentions that “the active expansion of the braid optionally may be assisted via inflation of a balloon catheter (not shown) reversibly disposed within apparatus 10”, it further explains that “expansion without use of a balloon catheter is preferred such that flow is never occluded during deployment”.

Balloon-type valvuloplasty devices that enable blood flow have also been described. For example, US 20050090846 describes various balloon configurations that enable flow through or around the balloon when inflated. Although, in theory, such configurations provide advantages over standard balloon valvuloplasty devices, the use of a balloon limits the force that can be applied to the valve tissue.

Such attempts to address the limitations of prior art balloon valvuloplasty devices have, to date, yielded partial solution and as such, there remains a need for non-balloon valvuloplasty device that can provide the tissue forces necessary for reshaping calcified valves while at the same time enable blood flow through the valve during the procedure.

Thus, according to one aspect of the present invention there is provided a device for resizing/reshaping a tissue opening.

As used herein, the phrase “resizing/reshaping a tissue opening” refers to changing the size (e.g. diameter) or shape of a tissue opening. Such resizing or reshaping can be elastic or plastic depending on the tissue. For example, in the case of stenotic heart valves in which the heart valve tissue is calcified and the movement of the leaflets and orifice size are reduced, resizing/reshaping can permanently modify heart valve morphology and orifice size (plastic resizing/reshaping).

As used herein, the phrase “tissue opening” refers to an opening through a tissue structure in a body of a mammal such as a human. The lumen of a vessel, duct, or other tubular or saclike organ or tissue structure and the orifice of a valve (e.g. heart valve) or sphincter are all tissue openings.

In one embodiment, the device of the present invention includes a cage structure which is positionable within the tissue opening and is radially expandable therein.

As used herein, the phrase “cage structure” refers to any fully or partially enclosed structure formed from wires, struts/beams and the like. The cage structure can be constructed by soldering or weaving wires (e.g. Nitinol wires) or but etching or cutting (e.g. laser cutting) the structure from a tube. In any case, the cage structure constructed in accordance with the teachings of the present invention is configured such that when fully expanded, the spaces between adjacent wires, struts etc enable a flow of fluid therethrough. In cases where the fluid is blood, the cage structure is constructed with spaces of a size that enables blood flow while limiting shear-induced hemolysis. Examples of various cage structure configurations are provided hereinbelow.

The cage structure can be expanded using various approaches including, for example, wires or other mechanical elements.

According to one preferred embodiment of the present invention, the cage structure is at least partially expanded via inflation of at least one balloon positioned within the cage structure.

In such a configuration, the balloon is positioned such that when it is fully inflated, the cage structure fully expands while maintaining flow-through capabilities, i.e. the fully expanded balloon does not completely block flow through the spaces of the cage structure.

For purposes of illustration, the Figures described below relate to a valvuloplasty embodiment of the present device which is preferably used with percutaneous delivery systems. However, it is to be understood that the present invention also encompasses configurations of the present device suitable for use with trans-apical delivery systems (via minimally invasive procedures) and in resizing/reshaping of other types of tissue openings.

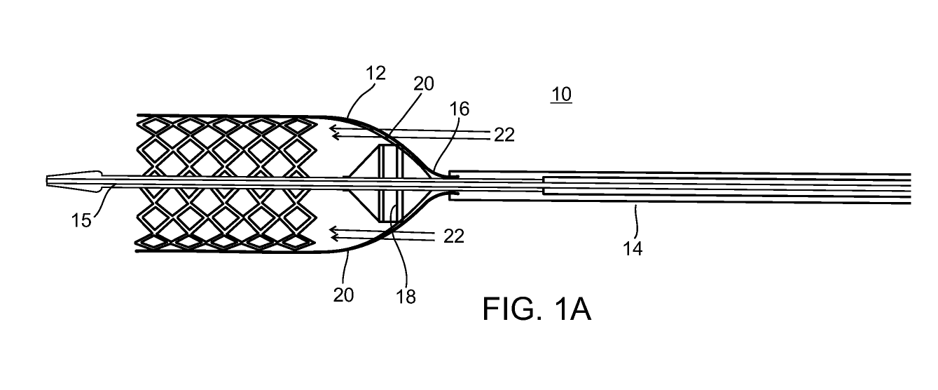

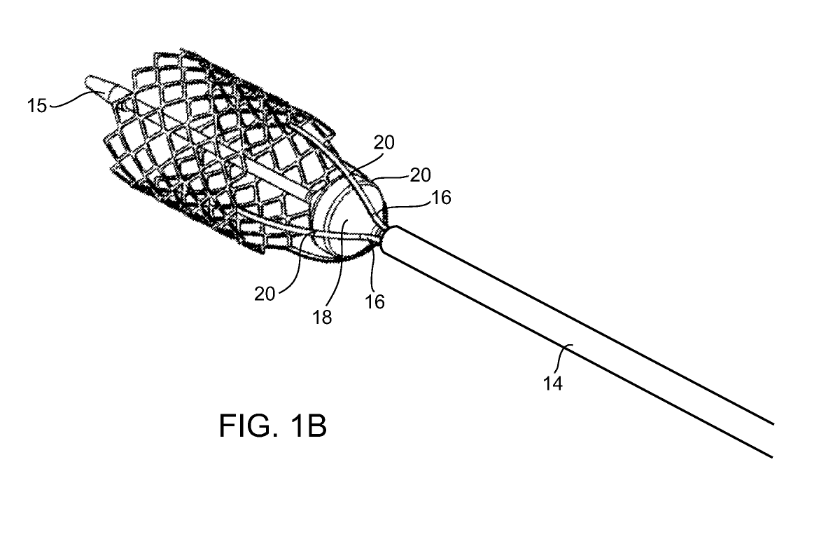

FIGS. 1a-4d illustrate several valvuloplasty configurations of the device of the present invention which is referred to herein as device 10.

Device 10 of FIGS. 1a-b includes cage structure 12 (also referred to herein as cage 12) which in this configuration is constructed from a mesh tube which is attached at a proximal end 16 thereof via struts 20 to a delivery catheter 14 (mounted on a guide wire 15). Cage structure 12 of FIGS. 1a-b is shown in a fully expanded state.

Cage 12 can be constructed by laser cutting a Nitinol tube and thermally treating it (450-550° C.) to obtain a predefined expended diameter and shape. Each strut can be 0.1-1.0 mm wide and 0.1-0.5 mm thick. The initial diameter of the tube (before cutting and shaping) can be 3-15 mm, and the length (proximal to distal) 20-50 mm An attachment ring can be formed at the proximal end during cutting; the ring can then be glued to catheter 14 during assembly of cage 12 thereupon.

Alternative configurations of cage structure 12 can be constructed from soldered or glued struts and the like.

When fully expanded, cage 12 has a diameter ranging from 20-40 mm; when compressed (and sheathed), cage 12 has a diameter ranging from 3-8 mm.

Device 10 also includes a balloon 18 (shown fully expanded) which is preferably a non-compliant or semi-compliant balloon. In the configuration of FIGS. 1a-b, balloon 18 is attached to delivery catheter 14 within cage 12, at a proximal portion thereof. Balloon 18 can be inflated via any fluid, including air, gas, water saline and the like. Typical size ranges for balloon 18 are 2-5 mm when deflated and up to 8-20 mm when inflated. Balloon 18 is preferably a non-compliant polyamide balloon capable of expanding to a diameter of 20 mm and holding a pressure of up to 8 atmospheres. The balloon can also include radio-opaque marker rings at both ends and at mid length. Balloon 18 is preferably bonded to delivery catheter 14 which includes the fluid conduits (not shown) supplying the balloon with a fluid (e.g. saline) contained in an external reservoir.

Inflation of balloon 18 applies an outward force on struts 20 of cage structure 12 and forces it to radially expand via elastic deformation. Radial expansion of cage structure 12 applies a force to the tissue surrounding the tissue opening and forces such tissue outwards. Calculation of the forces applied to the tissue and those acting on cage structure 12 and balloon 18 are provided in the Examples section which follows.

As is shown in FIGS. 1a-b, positioning of balloon 18 within cage structure 12 enables full expansion of cage structure 12 but at the same time ensures that a fully expanded balloon 18 does not fully occupy the interior space of the expanded cage structure 12. This ensures that when device 10 is fully expanded, it still supports flow therethrough (shown by arrows 22). This feature of device 10 of the present invention is advantageous when performing valvuloplasty procedures since it enables the physician to maintain a resizing/reshaping force against the valve leaflets for extended time periods.

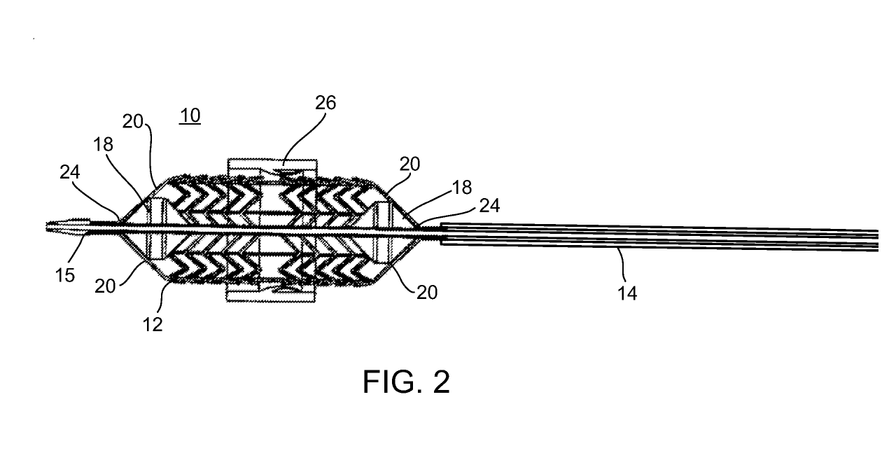

FIG. 2 illustrates another embodiment of a valvuloplasty device 10, shown positioned within valve 26. In this embodiment, cage structure 12 forms a bilaterally symmetrical torpedo-like structure with two tapering ends 24, at least one of which being permanently attached to delivery catheter 14.

Device 10 of FIG. 2 includes two balloons 18 each residing within an end portion of cage structure 12. Each side of this configuration of device 10 is essentially identical to device 10 of FIGS. 1a-b.

This configuration of device 10 is expanded in a manner similar to the configuration of FIGS. 1a-b and provides the same advantages in as far as flow. However, since this configuration of device 10 employs two balloons 18 and a closed bilaterally tapering structure, it enables application of a greater force on valve 26.

Since radial expansion of a bilaterally symmetrical cage structure 12 (such as the cage shown in FIG. 2) can result in end-to-end shortening of cage structure 12 (along catheter 14), when needed, device 10 preferably includes a mechanism for compensating for such a change in length.

Such a mechanism can be built into cage structure 12 or provided at the cage 12—delivery catheter 14 interface.

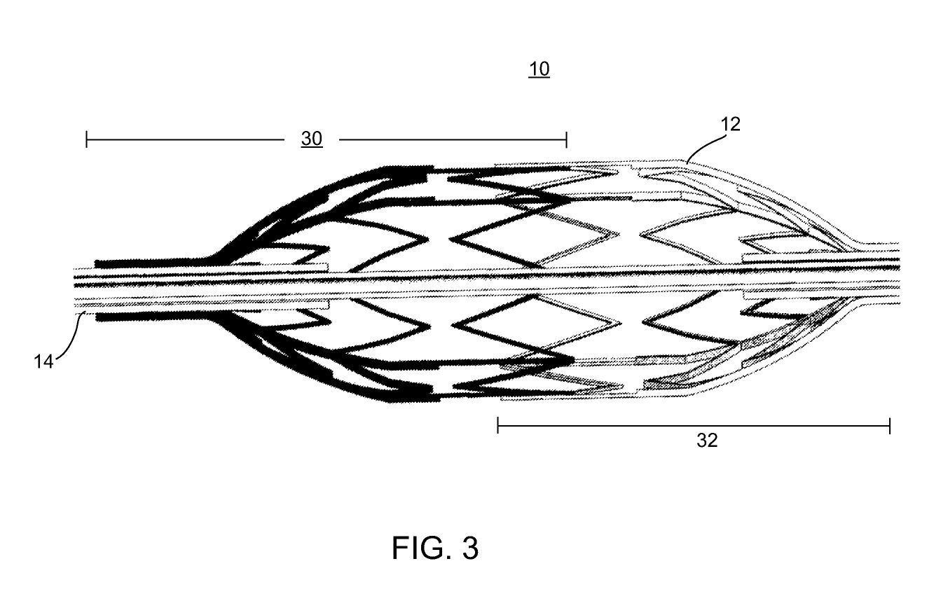

FIG. 3 illustrates one cage 14 configuration that enables length compensation during expansion (balloons 18 not shown). In this configuration, cage structure 12 is fabricated from two separate halves (designated 30 and 32), each being capable of sliding along each other when they shorten (along the axis of catheter 14) during expansion.

Such a configuration is constructed by laser cutting each of halves 30 and 32 from a Nitinol tube and thermally treating the cut halves to obtain the final shape and dimensions. Halves 30 and 32 can then be separately glued to a tube or tubes that are assembled over catheter 14 and fixed to it via, for example, glue.

Such a configuration provides compensation for a change in length while not substantially compromising the integrity of cage 12 or its ability to apply a force on the valve tissue. As is further described hereinunder, such a configuration also provides additional sizing features.

Compensation for a change in length can also be provided in the struts/wires of cage 12. For example, struts 20 of cage 12 can be fabricated with elastic s-shaped sections which stretch during expansion thereby compensating for expansion.

Compensation can also be effected by enabling one end 24 of cage structure 12 to slide along delivery catheter 14. Such sliding ensures that when cage 12 is expanded, free end 24 slides in (towards the opposite end 24) along delivery catheter thereby compensating for structural changes resulting from radial expansion.

Such a configuration also includes a mechanism (e.g. wire activated lock) for locking and unlocking end 24 to catheter 14.

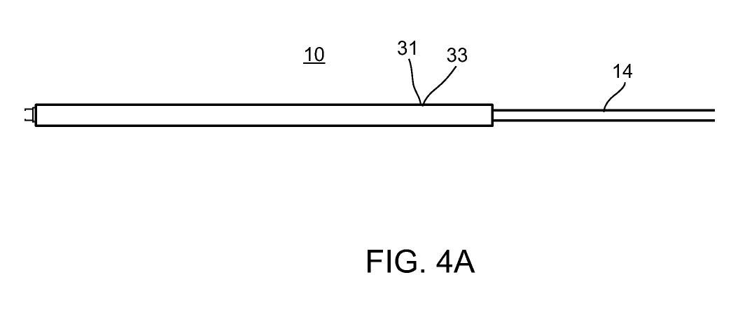

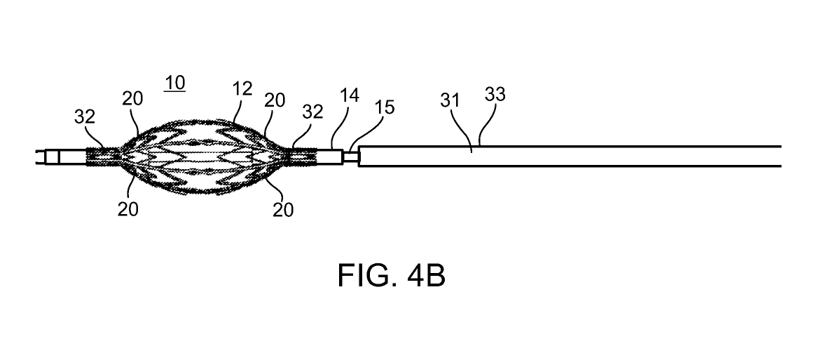

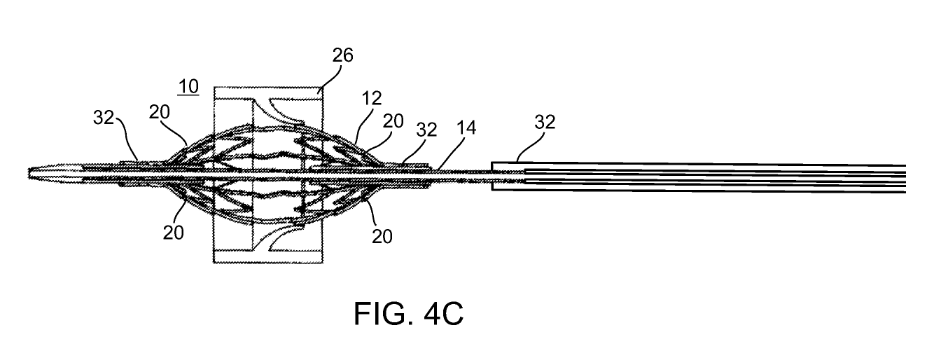

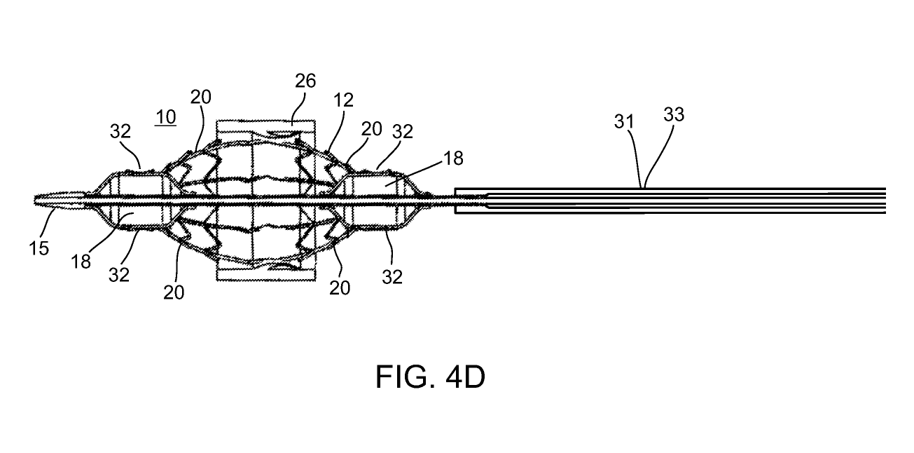

FIGS. 4a-d illustrate yet another configuration of device 10. In this configuration expansion of cage structure 12 is effected via two separate mechanism, the first radial expansion is effected via elastic deformation of cage structure, and the second via an expansion mechanism.

The first radial expansion phase is illustrated in FIGS. 4a-b. A cage structure having a preset elastic expansion phase is packed into a compressive element 31 (e.g. sheath 33 covering cage structure shown in FIG. 4a). Upon removal of element 31, (effected by, for example, pulling back sheath 33), cage structure assumes a first radial expansion state via elastic deformation (FIG. 4b). Following such elastic deformation the diameter of cage structure 12 can be anywhere from 8 to 20 mm.

The first expansion phase can be effected within the body but out of the valve region (e.g. in the aorta or ventricle), although in most cases, such expansion preferably takes place following positioning of cage 12 region of catheter 14 in the tissue opening (orifice of valve 26—FIG. 4c).

Since the first expansion phase involves a change in length of cage structure 12 (to accommodate for radial expansion), device 10 or specifically struts 20 of cage structure 12 preferably employ any of the length compensating mechanism described above.

The second expansion phase is effected via mechanical expansion of cage structure 12. Various approaches utilizing wire- or fluid-activated expansion mechanisms are described hereinabove. Preferably, the second expansion phase is effected using one or more balloons 18.

In the configuration of FIGS. 4a-d, end portions 32 of struts 20 are attached directly to balloons 18 which are in turn attached to catheter 14. Inflation of balloons 18 lifts struts 20 (moves them outward), thereby radially expanding end portions of cage structure 12 without elastically deforming struts 20 or cage structure 12.

Such radial expansion is different than that described above with respect to FIGS. 1a-b. In the configuration of FIGS. 1a-b, inflation of the balloon applies a force to struts 20 which results in the outward deflection of struts 20 and the elastic deformation thereof.

Such bi-phasic expansion is advantageous since it enables tight packing of cage structure 12 and balloons 18 within a delivery catheter to a final size of 12 French (F) or less, while at the same time it enables substantial radial expansion of cage structure (more than 40 mm) without considerable deformation. Since elastic deformation generates a counter force (on balloons 18), minimizing deformation ensures that counter forces are minimized resulting in more efficient force transmittal to the cage and in turn to the tissue.

It will be appreciated that although the balloon expansion (2^{nd} phase) of cage structure 12 is preferred, mechanical expansion using wire actuated expansion mechanisms (replacing balloons 18) is also envisaged herein.

Several types of mechanical expansion approaches can be used by the present invention.

For example, a distal end of cage structure (fitted over an external hypotube) can be pulled towards a fixed proximal end using a dedicated wire, or a guide wire fitted with a stop. Such pulling compresses cage 12 longitudinally while expanding it radially.

Alternatively, two expansion structures, each fitted at an end of cage 12 can be used to ‘jack up’ end portions of cage 12 in a manner similar to balloons 18. These expansion structures are fitted over the catheter and are actuated by a dedicated wire which when pulls compresses each of the structure longitudinally and as a result causes radial expansion of these structure and ‘jacking’ of end portions of cage 12.

Thus, the present invention provides a device for resizing/reshaping a tissue opening which is designed for enabling flow through the device during a tissue opening resizing/reshaping procedure.

The present device can also incorporate a valve mechanism (disposed within cage structure 12) in order to prevent back flow through the device (i.e. to ensure directional flow out of the heart). Such a valve can use a single leaf that moves with the blood flow in (to stop flow) and out (to allow flow). Valve functionality can also be achieved without employing moving parts. For example, an element positioned within device 10 can be designed to apply drag to blood flowing in one direction but not another. Thus, blood flow in one direction will be much higher than the other, enabling substantial blood flow from the LV to the aorta, and minimal flow in the opposite direction.

The control of blood flow in both direction can also be achieved by utilizing balloons 18 which are shaped to impede (apply drag forces) flow from the aorta to the LV and yet not substantially impede flow from the LV to the aorta. An example of such a configuration can employ wedge-shaped balloon with the point facing the LV.

In order to ensure accurate and effective force transmission to the valve tissue, cage structure can include a saddle-shaped area (when expanded) at the region of contact with the tissue. Such a concaved region ensures that the tissue

The design of the present device provides several advantages in valvuloplasty procedures:

(i) It enables flow of blood through the device during the procedure;

(ii) It provides controllable expansion while enabling effective transmittal of force to the valve tissue

(iii) It ensures a non-slip interface with valve tissue

(iv) It enables blood flow in LV-aorta direction while restricting flow in the opposite direction.

Since the device of the present invention is used by the physician prior to prosthetic valve placement, the present device can also be used to mark the valve (e.g. valve annulus) with a contrast agent and thus facilitate accurate guiding and positioning of the prosthetic valve. Examples of contrast agents include water soluble or water insoluble radiopaque contrast agents. Examples of water soluble radiopaque contrast agents include metrizamide, iopamidol, iothalamate sodium, iodomide sodium, and meglumine Examples of water insoluble radiopaque contrast agents include metals and metal oxides dyes or microsphere suspensions, gels and the like.

Such contrast agents can be delivered into annulus or leaflet tissue via a cannula or needle mounted on a catheter carrying the present device. The catheter or device can also include a reservoir filled with the contrast agent. Alternatively, cage structure 12 can be partially covered with a polymeric sleeve or sheath impregnated/coated with a radio-opaque marker. Such a sleeve can be used to blot transfer the marker to the tissue upon contact between cage structure 12 and valve tissue. Since device 10 of the present invention can be expanded and maintained in place for relatively long periods of time, it facilitates effective “tattooing” of the tissue with the marker. It will be appreciated that cage 12 can also be covered with a drug releasing coating/cover and used to release a drug to the valve tissue in order to reduce valve leaflet restenosis following a valvuloplasty procedure.

Since the device of the present invention can be accurately positioned within the valve and anchored therein (further described hereinbelow), it can also serve to accurately guide a delivery cannula or needle to the annulus or leaflet tissue.

The present device can also be utilized for determining a size and shape of a tissue opening such as a valve annulus.

One preferred approach for such size/shape determination utilizes a plurality of imaging markers (e.g. radio-opaque, fluorescent, RF, or ultrasound markers) arranged in a pattern on the cage structure. When the expandable member is positioned in the tissue opening and expanded, positioning of these markers with respect to each other can be used to determine the diameter of the cage structure and thus the diameter of the valve opening, and a cross sectional shape of the cage structure and thus a cross sectional shape of the tissue surrounding the opening.

The average size/diameter of the valve can alternatively be determined via longitudinal and/or radial movement of struts 20 during expansion.

For example, in the case of the device illustrated in FIG. 3, expansion of the cage structure leads to movement of halves 30 and 32 outward (away from each other). Thus, using markers positioned on struts of opposing halves, one can estimate a diameter of the cage structure based on a distance between such opposing markers.

Shape extraction also enables determination of a projection plane which is most perpendicular to the device and thus most perpendicular to the annulus. Information relating to the projection plane of the annulus can be used by a physician to subsequently deliver and accurately position a prosthetic valve.

Specific examples of such markers include gold markers which can be mechanically applied to structure 16 (via crimping) or glued or heat-fused thereto (point welding). Alternatively, a Barium Sulfate or Tungsten Carbide solution can be mixed into a polymer material used to coat the cage structure.

Size of valve annulus can also be determined using the volume/pressure data of balloons 18 of device 10 shown in FIGS. 1a-b. By plotting pressure versus volume of balloons 18 during inflation, one can identify initial contact between cage structure 12 and valve annulus tissue and use the pressure and volume data associated with that point to estimate the diameter of cage structure 12 and hence the diameter of the valve.

To enable accurate positioning within a specific zone of a tissue opening, the present device can also include a tissue engagement mechanism which ensures that the cage structure is correctly positioned within the desired zone of a tissue opening. This feature is particularly useful on cases where the tissue opening is defined by a narrow tissue zone (e.g. valve opening) since in such cases, positioning of the cage structure within the desired zone in the tissue opening and/or maintaining such positioning throughout a procedure can be difficult to achieve.

One proposed gripping structure can be constructed by covering only a middle portion of cage structure 12 with a sheath or sleeve.

The device of the present invention can also incorporate elements that can be used to score (notch) valve leaflet tissue. This feature is particularly useful in heart valve stenosis, where fibrosis of the valve leaflets may limit the opening size of the heart valve.

The scoring process is based on stress concentration principle wherein a very narrow point or edge applies force over a surface, local contact forces will rise dramatically as function of the “in contact” surface area.

One embodiment of a scoring configuration can be effected by constructing cage structure with thin wires (or struts) and then shaping them with a very narrow edge on an external side (facing the tissue). Such wires or struts would score or notch calcified leaflet tissue with application of minimal pressure.

Since the device of the present invention applies stent-like forces to the valve tissue, it can be used to estimate the clinical affect of a valve carrying stent deployed following valvuloplasty. For example, device 10 can be used to estimate future rhythm complications that may result from pressure/contact between a prosthetic valve and the bundle of His. Such possible rhythm complications can be analysed by providing device 10 with sensing electrodes and measuring outside the patient body any tissue electrical disturbances during deployment of device 10.

The device of the present invention can also incorporate a trap for trapping calcified fragments dislodged from valve tissue upon valvuloplasty. Such a trap, which is preferably deployed on the catheter proximal to cage structure 12 can be similar in design to embolic traps that are well known in the art.

As is mentioned hereinabove and illustrated in the Figures, the present device is mounted on a delivery catheter which forms a part of a system which facilitates positioning of the present device within a tissue opening and providing expansion functionalities.

Thus, according to another aspect of the present invention there is provided a system for resizing/reshaping a tissue opening.

The systems includes the present device fitted on, or integrated with a delivery catheter. The catheter can be any percutaneous delivery catheter, preferably, the catheter is an over-the-wire Valvuloplasty (or other) catheter with enhanced strength pushability, and a flexible distal tip. Depending on the configuration of the present device, the balloons used for expansion of the cage structure can be directly attached to, or formed with the catheter.

The present system further includes a control unit (not shown) which is connected to an extracorporeal end of the catheter. The control unit includes a Fluid Pressure generating device (e.g. pump) which is connected to conduits in the catheter via a standard catheter interface (as is utilized in the art).

The present system can be used for resizing/reshaping of a valve as follows.

A guidewire is inserted into a heart chamber via femoral, axial, radial or apical access and the catheter is positioned over the wire to a desired location. Once the catheter and mounted device are positioned an imaging C-arm is maneuvered over the site of imaging and used to ascertain that device is positioned in place, by imaging and identifying markers disposed on the device and/or catheter.

The cage structure is expanded as described above to a point where it contacts annulus tissue, the physician cab then maintain the device in this position for a predetermined time period or collapse the cage structure and re-expand it one or more additional times.

The physician then images the device within the annulus at one or more projection angles to identify the markers and determine the size/shape of the treated valve. Once imaging is complete, the cage structure is collapsed and the catheter is removed.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

An optimal device configuration was designed taking into consideration device packing diameter (for delivery), maximal radius of the cage for enabling application of force to the valve leaflets and annulus and the ability of the cage struts to carry the load of the force applied to the valve tissue.

The radial forces needed for effective valvuloplasty were determined by analyzing the forced applied by a balloon valvuloplasty device. Max pressure of a balloon valvuloplasty device (as measured at the pump) is about 5 atmospheres (atm). The semi-compliant valvuloplasty maintains 3.5-4 atm at maximal volume. Thus, the additional 1-1.5 atm represent the force applied by the tissue on the balloon and vise versa.

In order to provide such forces in a cage-like device having an N number of struts having a length of 25-60 mm, and maintain device integrity and packing diameter of 12-15 F or less (when used with percutaneous catheter delivery systems), one must consider the following:
1. Number of struts: shape optimization (as close to a circle as possible) per folded size—5-8 struts will create a shape that is close enough to a circle using 5-6 struts may allow minimization of the packed profile of the device and allow delivery through 12-15 F catheter, while maintaining desired integrity.
2. Expansion:
(i) Due to the differences in diameter between the packed and fully open configurations and the forces necessary for reshaping the valve tissue (about 0.5-2.5 kg of force per strut in 6 bars configuration) a two step strut expansion process is required. Initial opening of the struts is effected via elastic deformation (by releasing a sheath trapping the struts). The released struts are arced outward (radially) with a maximum deflection of 0.3-15 mm (to a device diameter of about 12-15 mm) The arced configuration allows the struts to carry the load of the force applied to the valve tissue.
(ii) Since a final device diameter of 18-28 mm is required in order to provide the reshaping force, balloons positioned at ends of the cage will be used to “jack up” the cage to its final diameter, balloon inflation pressure is in the range of 2-6 atm.
(iii) Balloon inflation adds 3-13 mm to the diameter of the cage without changing the configuration of the struts. A relatively low profile balloon can be used which can be tightly packed within a 12 F diameter (25 mm diameter balloon) or a 7-9 F diameter (10-13 mm diameter balloon).
3. The main advantage of using a hydraulic jacking approach (balloon) as opposed to a mechanical approach (cables) is that relatively large pressures (and as a result large forces) can be transferred along the 1.5 meter catheter with relatively small effect on the maneuverability of the catheter. Using a cable activated mechanical system to generate such forces (pull/push hypotubes in an “over the wire” system) may require a stiffer design and as result would substantially decrease system maneuverability.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

In the claims:

1. A device for resizing/reshaping a tissue opening comprising a cage structure being positionable within the tissue opening and being radially expandable therein via inflation of at least one balloon positioned within said cage structure, wherein the device enables flow of a biological fluid through the tissue opening when said at least one balloon is fully inflated and said cage structure is fully expanded and biased against a tissue defining the tissue opening.

2. The device of claim 1, wherein the tissue opening is a heart valve orifice and said biological fluid is blood.

3. The device of claim 1, wherein said cage structure is torpedo shaped.

4. The device of claim 3, wherein said at least one balloon includes two balloons positioned at an end portions of said torpedo shaped cage structure.

5. The device of claim 1, wherein said cage structure is radially expandable to a diameter of 20-40 mm

6. The device of claim 1, further comprising a valve for controlling flow of said biological fluid through the device.

7. The device of claim 1, where said at least one balloon is configured for reducing drag forces on flow of said biological fluid in one direction, while increasing said drag forces on said flow of said biological fluid in an opposite direction.

8. The device of claim 1, wherein the device is designed for compensating for changes in a length of said cage structure during radial expansion thereof.

9. The device of claim 8, wherein said cage structure includes elastic struts configured for compensating for changes in said length of said cage structure during radial expansion thereof.

10. The device of claim 1, wherein said cage structure is formed of longitudinal struts.

11. A device for resizing/reshaping a tissue opening comprising a cage structure being positionable within the tissue opening and being capable of a first radial expansion via an elastic structural change and a second radial expansion via mechanical expansion.

12. The device of claim 11, wherein said elastic structural change is effected by releasing a restraint maintaining said cage structure in a compressed state.

13. The device of claim 12, wherein said second radial expansion is effected by a mechanism being capable of radially expanding said cage structure without elastic deformation.

14. The device of claim 11, wherein said mechanism includes at least one balloon disposed within said cage structure.

15. The device of claim 11, wherein the device enables flow of a biological fluid through the tissue opening when said at least one balloon is fully inflated and said cage structure is fully expanded and biased against a tissue defining the tissue opening.

16. The device of claim 11, wherein said first radial expansion of said cage structure is up to a diameter of 20 mm

17. The device of claim 16, wherein said second radial expansion of said cage structure is up to a diameter of 40 mm

18-20. (canceled)

21. A method of resizing/reshaping a tissue opening comprising:
(a) positioning a cage structure within said tissue opening;
(b) expanding said cage structure within the tissue opening thereby biasing outward tissue defining the tissue opening while enabling biological fluid to flow through the tissue opening, thereby resizing/reshaping the tissue opening; and
(c) controlling said flow through said cage structure.

22-24. (canceled)

25. The method of claim 21, wherein (b) is effected via at least one balloon position within said cage structure.

26. The method of claim 21, wherein the tissue opening is a heart valve orifice and said biological fluid is blood.
